(19) United States
(12) Patent Application Publication
(10) Pub. No.: US 2011/0009729 A1
(43) Pub. Date: Jan. 13, 2011

(54) APPARATUS FOR MEASURING PHYSIOLOGICAL SIGNALS

(75) Inventors: Seungchul SHIN, Daejeon (KR); Inbum Lee, Daejeon (KR); YongWon Jang, Daejeon (KR); Yoon Seon Song, Daejeon (KR); Seunghwan Kim, Daejeon (KR); Seon Hee Park, Daejeon (KR)

Correspondence Address: AMPACC Law Group, PLLC, 6100 219th Street SW, Suite 580, Mountlake Terrace, WA, 98043, US

(73) Assignee: Electronics and Telecommunications Research Institute, Daejeon (KR)

(21) Appl. No.: 12/689,041

(22) Filed: Jan. 18, 2010

(30) Foreign Application Priority Data
Jul. 8, 2009 (KR) 10-2009-0062207

(51) Int. Cl. A61B 5/04

(52) U.S. Cl. 600/391; 600/393

(57) ABSTRACT

Provided is an apparatus for measuring physiological signals by distributing modules according to functions. The physiological signal measuring apparatus includes first and second detection electrodes, a physiological signal detection module, a power supply module, and a connection line. The first and second detection electrodes are adhered to a target skin to detect physiological signals. The physiological signal detection module is installed at the first detection electrode to generate physiological signal data from the physiological signals detected by the first and second detection electrodes. The power supply module is installed at the second detection electrode to supply power to the physiological signal detection module. The connection line connects the physiological signal detection module and the power supply module.

### CROSS-REFERENCE TO RELATED APPLICATIONS

This U.S. non-provisional patent application claims priority under 35 U.S.C. §119 of Korean Patent Application No. 10-2009-0062207, filed on Jul. 8, 2009, the entire contents of which are hereby incorporated by reference.

### BACKGROUND OF THE INVENTION

The present invention disclosed herein relates to physiological signal measuring apparatuses, and more particularly, to physiological signal measuring apparatuses that can measure physiological signals more conveniently by distributing modules according to functions.

As an interest in health increases, ubiquitous healthcare, which merges medical technology and IT technology together, is attracting much attention. The ubiquitous healthcare collects heath-related physiological signals everywhere in real time, transmits the physiological signals to a healthcare service center, and checks health conditions to provide continuous healthcare and disease management services.

The ubiquitous healthcare may include a physiological signal measuring apparatus capable of easily measuring health-related physiological signals (e.g., blood pressure, blood sugar, body weight, electrocardiogram, quantity of motion, breath, and temperature) everywhere, a network for transmitting the measured physiological signals through various wired/wireless communication technologies, an information collection and management system for collecting the transmitted information, and an application service for analyzing the collected information to provide various services.

In the ubiquitous healthcare, the physiological signal measuring apparatus includes an electrode attached to the skin to measure a bio-potential, a power supply unit, and a controller for interpreting and processing a physiological signal measured by the electrode.

In the case of a typical body-attached physiological signal measuring apparatus, all the components are disposed in one package. Accordingly, the size and weight of a device applied to a portion of the human body increase, thus increasing a foreign-body sense and an inconvenience in use. Also, an electrode with a stronger adhesive force must be used to support the weight of the apparatus, which may cause skin troubles. Also, the typical body-attached physiological signal measuring apparatus is inconvenient to use, particularly in the case of clinic patients or chronic patients requiring the long-time measurement of physiological signal.

### SUMMARY OF THE INVENTION

Embodiments of the present invention provide physiological signal measuring apparatuses that can measure physiological signals more conveniently.

The objects of the present invention are not limited to the aforesaid, and other objects not described herein will be clearly understood by those skilled in the art from descriptions below.

Embodiments of the present invention provide apparatuses for measuring physiological signals, including: first and second detection electrodes adhered to a target skin to detect physiological signals; a physiological signal detection module installed at the first detection electrode to generate physiological signal data from the physiological signals detected by the first and second detection electrodes; a power supply module installed at the second detection electrode to supply power to the physiological signal detection module; and a connection line connecting the physiological signal detection module and the power supply module.

The details of other embodiments are included in the detailed description and the drawings.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 illustrates an outline structure of a physiological signal measuring apparatus according to an exemplary embodiment of the present invention;

FIG. 2 illustrates a connection between a detection electrode and a module in the physiological signal measuring apparatus according to an exemplary embodiment of the present invention;

FIG. 3 illustrates a structure of each of the distributed modules of the physiological signal measuring apparatus according to an exemplary embodiment of the present invention;

FIG. 4 illustrates an example of the use of the physiological signal measuring apparatus according to an exemplary embodiment of the present invention;

FIG. 5 illustrates an outline structure of a physiological signal measuring apparatus according to another exemplary embodiment of the present invention;

FIG. 6 illustrates a structure of each of the distributed modules of the physiological signal measuring apparatus according to another exemplary embodiment of the present invention;

FIG. 7 illustrates an example of the use of the physiological signal measuring apparatus according to another exemplary embodiment of the present invention;

FIG. 8 illustrates an outline structure of a physiological signal measuring apparatus according to still another exemplary embodiment of the present invention;

FIG. 9 illustrates a structure of each of the distributed modules of the physiological signal measuring apparatus according to still another exemplary embodiment of the present invention;

FIG. 10 illustrates an example of the use of the physiological signal measuring apparatus according to still another exemplary embodiment of the present invention; and

FIGS. 11 to 14 illustrate various connections between the modules in the physiological signal measuring apparatus according to exemplary embodiments of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Like reference numerals refer to like elements throughout the specification.

In the following description, the technical terms are used only for explaining specific exemplary embodiments while not limiting the present invention. The terms of a singular form may include plural forms unless otherwise specified. The meaning of “include,” “comprise,” “including,” or “comprising,” specifies a property, a region, a fixed number, a step, a process, an element and/or a component but does not exclude other properties, regions, fixed numbers, steps, processes, elements and/or components.

A physiological signal measuring apparatus according to exemplary embodiments of the present invention can measure physiological signals that are generated due to a physiological potential difference of the human body. Examples of the physiological signals include electrocardiogram (ECG), electromyogram (EMG), electroencephalogram (EEG), galvanic skin reflex (GSR), and electrooculography (EOG).

The physiological signal measuring apparatus according to the exemplary embodiments of the present invention includes a plurality of detection electrodes, functional modules installed respectively at the detection electrodes, and connection lines connecting the functional modules. Examples of the functional modules include a physiological signal detection module for detecting analog signals of physiological potentials, a power supply module for supplying power, a physiological signal transmission module for transmitting detected physiological signals, and a physiological signal storage module for storing the detected physiological signals.

In exemplary embodiments of the present invention, the modules of the physiological signal measuring apparatus include a display unit for displaying the operation states of the modules and a control unit for allowing a user to control the modules.

In exemplary embodiments of the present invention, the modules of the physiological signal measuring apparatus may include a plurality of module connection ports for connection with other modules. In exemplary embodiments of the present invention, the module connection ports may be used by converting the input/output of physiological signal data according to uses, and power may be supplied through the module connection ports.

Hereinafter, physiological signal measuring apparatuses according to exemplary embodiments of the present invention will be described in detail with reference to the accompanying drawings.

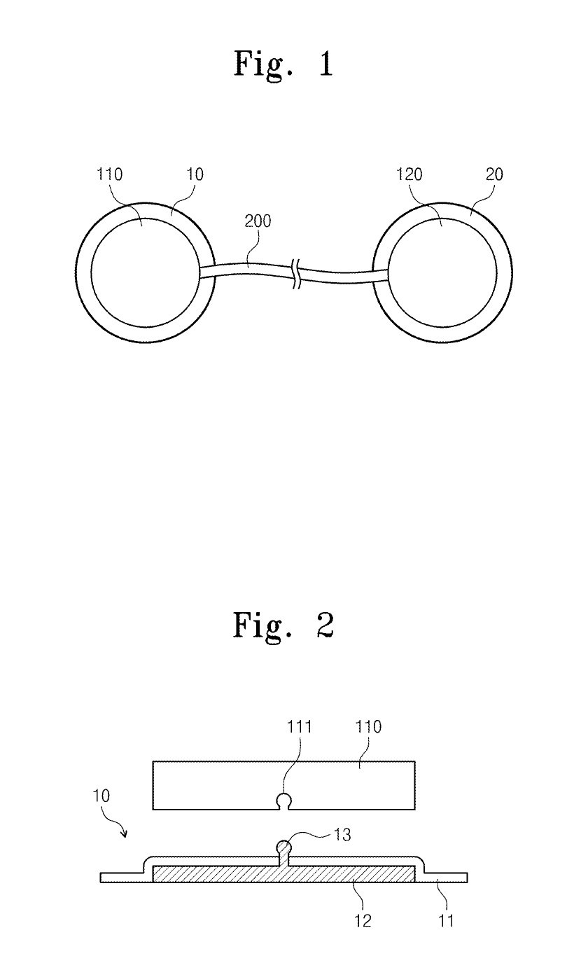

FIG. 1 illustrates an outline structure of a physiological signal measuring apparatus according to an exemplary embodiment of the present invention.

Referring to FIG. 1, a physiological signal measuring apparatus according to an exemplary embodiment of the present invention includes a first detection electrode 10, a second detection electrode 20, a physiological signal detection module 110 installed at the first detection electrode 10, and a power supply module 120 installed at the second detection electrode 20.

In the measurement of a physiological signal, the first and second detection electrodes 10 and 20 may be adhered to the skin of a human body to detect a physiological signal (i.e., a physiological potential) generated from the human body. The first and second detection electrodes 10 and 20 may be disposable electrodes, and may be connected/separated respectively to/from the physiological signal detection modules 110 and 120.

The physiological signal detection module 110 detects an analog signal of a physiological signal and generates physiological signal data of a digital signal converted from the physiological signal.

The power supply modules 120 supplies power to the physiological signal detection module 110.

Each of the physiological signal detection module 110 and the power supply module 120 may include a display unit for displaying the operation state of the corresponding module, and may have a control unit (e.g., a switch or a button) for allowing the user to control the corresponding module.

A connection line 200 connects the modules 110 and 120 installed respectively to the detection electrodes 10 and 20. The connection line 200 may include a data line for transmitting physiological signal data and a power line for supplying power. The connection line 200 may be connected to module connection ports of the modules 110 and 120 through connectors connected to both ends thereof. Also, the connection line 200 may be fixed to the modules 110 and 120 by soldering. The connection line 200 may have flexibility and elasticity for enabling the user to act freely in the measurement of physiological signals.

FIG. 2 illustrates a connection between the detection electrode and the module in the physiological signal measuring apparatus according to an exemplary embodiment of the present invention.

Referring to FIG. 2, in the physiological signal measuring apparatus, the detection electrode 10 includes an adhesive pad 11, a conductive electrode 12, and a module connection unit 13.

The adhesive pad 11 may be formed of nonconductive or insulating material. Examples of the conductive or insulating material include paper and insulating resin. Also, the adhesive pad 11 may have the shape of a circle or a polygon such as a quadrangle.

The conductive electrode 12 is fixed at the center of one surface of the adhesive pad 11, and has the module connection unit 13 connectable with the corresponding module. The module connection unit 13 may have a protrusion (i.e., a snap) protruding from the conductive electrode 12, and may pierce the center of the adhesive pad 11. For example, the conductive electrode 12 may be formed of titanium (Ti), aurum (Au), platinum (Pt), or Ag/AgCl.

The physiological signal detection module 110 attached at the detection electrode 10 includes a printed circuit board (PCB) mounted with various integrated circuits and a module case covering the PCB. The integrated circuits may be mounted on a flexible substrate in order to reduce the inconvenience to the user and provide more stable adhesion to the uneven skin surface in the measurement of physiological signals. The module case may have a connection groove 111 for connection with the detection electrode 10. That is, the module connection unit 13 of the detection electrode 10 may be fixed into the connection groove 111 of the physiological signal detection module 110.

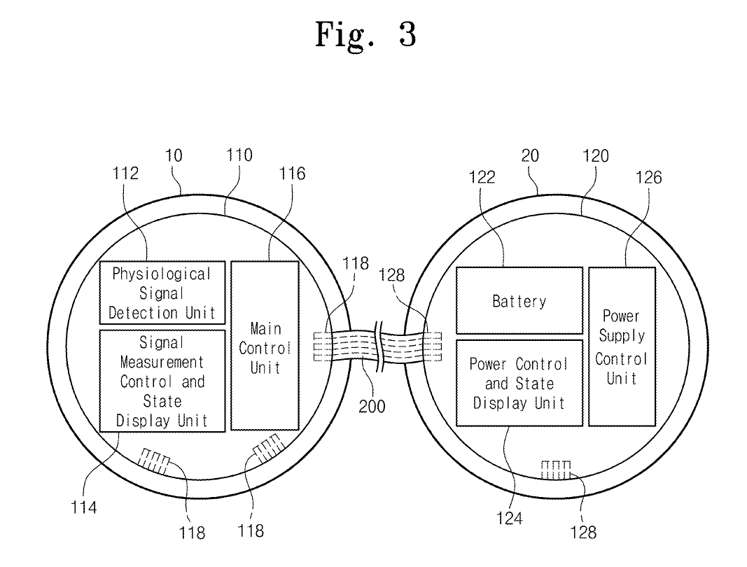

FIG. 3 illustrates a structure of each of the distributed modules of the physiological signal measuring apparatus according to an exemplary embodiment of the present invention.

Referring to FIG. 3, the physiological signal detection module 110 includes a physiological signal detection unit 112, a signal measurement control and state display unit 114, a main control unit 116, and one or more module connection ports 118.

The physiological signal detection unit 112 includes an analog signal processing unit, an analog-to-digital (A/D) converting unit, and a digital signal processing unit. The A/D converting unit may include an amplifier for amplifying minute electrical signals detected by the first and second detection electrodes 10 and 20 and a filter for removing a noise generated in the measurement of physiological signals. The A/D converting unit converts an analog physiological signal, received from the analog signal processing unit, into digital physiological signal data. The digital signal processing unit processes the physiological signal data, received from the A/D converting unit, through digital operation processes (e.g., a Fast Fourier Transform (FFT) operation, a differential operation, and an average operation).

The signal measurement control and state display unit 114 may control physiological signal detection according to the user's command, and displays an operation state of the physiological signal detection module 110. Also, the signal measurement control and state display unit 114 may display physiological signal data, received from the physiological signal detection unit 112, to the user.

The main control unit 116 is connected to the physiological signal detection unit 112, the signal measurement control and state display unit 114, and the module connection ports 118 to control physiological signal detection, power connection and data communication with other modules.

The module connection port 118 is connected to the connection line 200 to provide electrical connection between the modules. The module connection port 118 includes a power port and/or a data input/output port (e.g., an RS 232 port and a USB port) for serial communication of physiological signal data. Also, power and physiological signal data may be inputted/outputted through the module connection ports 118.

Also, the physiological signal detection module 110 may have a plurality of module connection ports 118 for connection with a plurality of other modules.

The power supply module 120 includes a battery 122, a power control and state display unit 124, a power supply control unit 126, and one or more module connection ports 128. For example, the power supply module 120 may have a disposable battery or a rechargeable battery. The user may control the power supply module 120 through the power control unit 124, and a power supply state and a residual battery capacity may be displayed on the state display unit 124. The connection line 200 is connected to the module connection port 128, and power is supplied through the connection line 200 to the physiological signal detection module 110.

The physiological signal detection module 110 and the power supply module 120 are electrically connected through the connection line 200 having connectors connected to both ends thereof. The connectors may be attachable/detachable to/from the module connection ports 118 and 128 in a snap configuration.

Also, the power supply module 120 may have a plurality of module connection ports 128 for connection with a plurality of other modules.

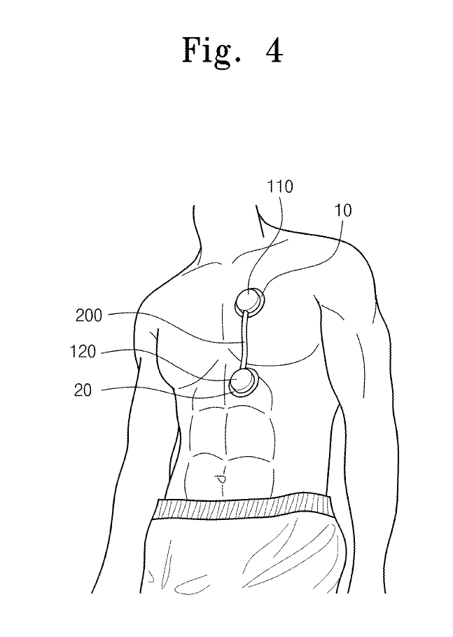

FIG. 4 illustrates an example of the use of the physiological signal measuring apparatus according to an exemplary embodiment of the present invention. As an exemplary embodiment of the present invention, a description will be given of an exemplary case of using the physiological signal measuring apparatus to measure an electrocardiogram (ECG).

Referring to FIG. 4, the physiological signal measuring apparatus can measure a 1-channel ECG when the first and second detection electrodes 10 and 20, at which the physiological signal detection module 110 and the power supply module 120 are installed, are attached to a heart portion of the human body. That is, when the physiological signal measuring apparatus is attached and the modules are operated, a physiological potential generated according to the shrinkage and expansion of heart muscles can be measured and a physiological signal can be displayed on the state display unit of the physiological signal detection module 110.

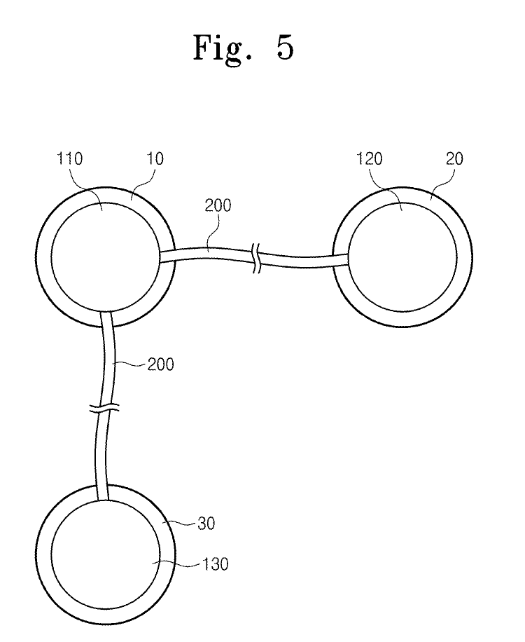

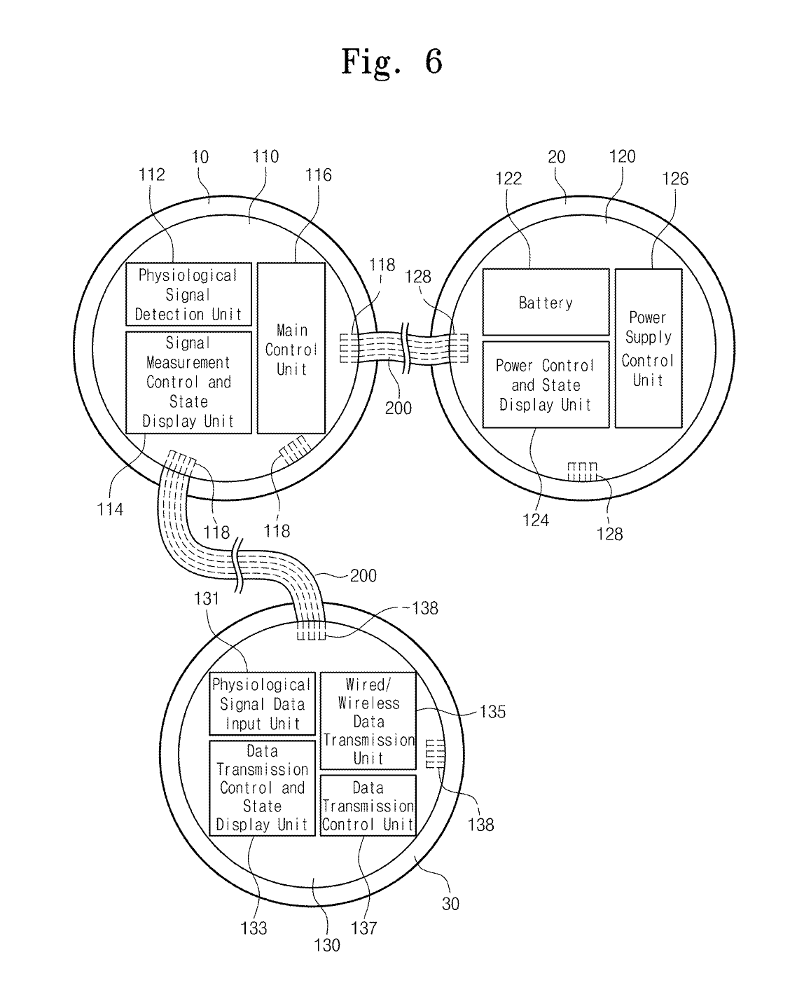

FIG. 5 illustrates an outline structure of a physiological signal measuring apparatus according to another exemplary embodiment of the present invention. FIG. 6 illustrates a structure of each of the distributed modules of the physiological signal measuring apparatus according to another exemplary embodiment of the present invention.

Referring to FIGS. 5 and 6, a physiological signal measuring apparatus according to another exemplary embodiment of the present invention includes first, second and third detection electrodes 10, 20 and 30, a physiological signal detection module 110, a power supply module 120, and a physiological signal transmission module 130.

The first, second and third detection electrodes 10, 20 and 30, the physiological signal detection module 110, and the power supply module 120 according to the exemplary embodiment of FIG. 5 are substantially identical to those according to the exemplary embodiment of FIG. 1, and thus their description will be omitted for conciseness.

The physiological signal transmission module 130 transmits physiological signal data, which are generated by the physiological signal detection module 110, to other device through wired/wireless communication. For example, the physiological signal detection module 130 may have a short-range wireless communication device (e.g., RF, wireless RAN, Bluetooth, and/or Zigbee) to transmit measured physiological data to an external network through wireless communication.

The physiological signal transmission module 130 is electrically connected through a connection line 200 to the physiological signal detection module 110. The connection line 200, connecting the physiological signal transmission module 130 and the physiological signal detection module 110, includes a data line for communicating physiological signal data and a power line for supplying power. That is, the physiological signal transmission module 130 may receive physiological signal data and power from the physiological signal detection module 110 through the connection line 200. Meanwhile, the physiological signal transmission module 130 may be connected through the connection line 200 to the power supply module 120, and may be connected to both the physiological signal detection module 110 and the power supply module 120.

Specifically, the physiological signal transmission module 130 includes a physiological signal data input unit 131, a data transmission control and state display unit 133, a wired/wireless data transmission unit 135, a data transmission control unit 137, and one or more module connection ports 138.

The physiological signal data input unit 131 receives physiological signal data generated by the physiological signal detection module 110. The received physiological signal data may be transmitted through the wired/wireless data transmission unit 135 to external devices (e.g., PCs, networks and PDAs).

The data transmission control and state display unit 133 may control a data transmission and a data transmission mode according to a user's command, and displays a data transmission state and an operation state of the physiological signal transmission module 130.

A connection line 200 is connected to the module connection port 138 to provide electrical connection between the modules. The module connection port 138 includes a data input/output port (e.g., an RS 232 port or a USB port) for serial communication of physiological signal data and/or a power port. Power and physiological signal data may be inputted through the module connection port 138 from the physiological signal detection module 110. Also, the physiological signal transmission module 130 may have a plurality of module connection ports 138. If the physiological signal transmission module 130 has a plurality of module connection ports 138, it may be electrically connected to other modules as well as the physiological signal detection module 110, unlike the illustration of FIG. 6.

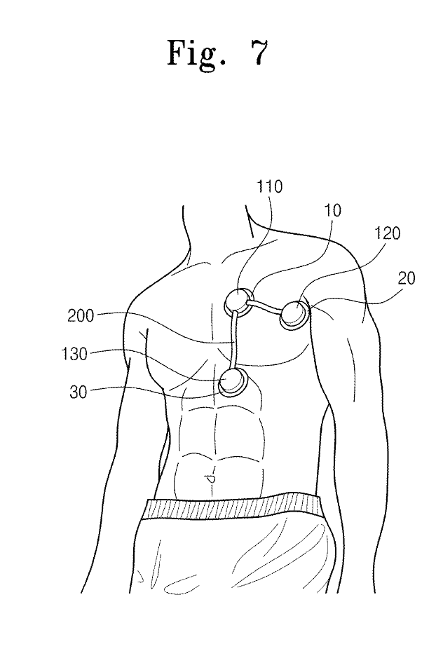

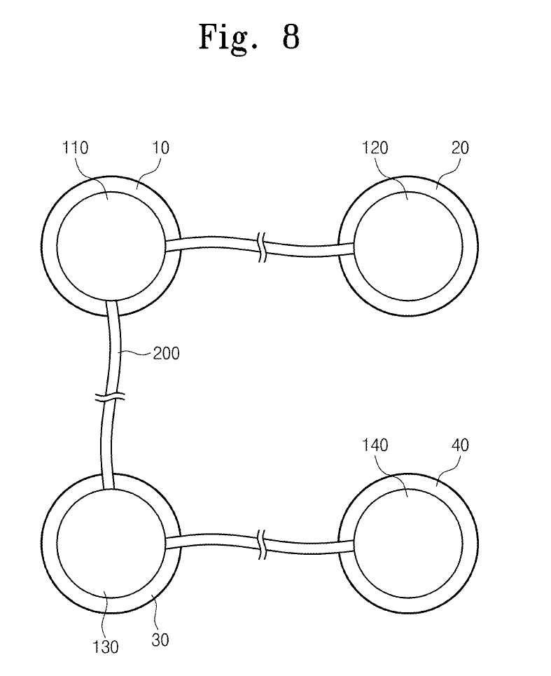

FIG. 7 illustrates an example of the use of the physiological signal measuring apparatus according to another exemplary embodiment of the present invention.

Referring to FIG. 7, the physiological signal measuring apparatus can measure a 2-channel ECG when the first, second and third detection electrodes 10, 20 and 30, at which the physiological signal detection module 110, the power supply module 120 and the physiological signal transmission module 130 are installed, are attached to a heart portion of the human body. Herein, the first detection electrode 10, at which the physiological signal detection module 110 is installed, may be adhered around an SA node that serves as a source for generating a heart pulsation pulse.

That is, when the physiological signal measuring apparatus is attached to the skin of the human body and the modules are operated, a physiological potential generated according to the shrinkage and expansion of heart muscles can be measured and a physiological signal can be displayed on the state display unit of the physiological signal detection module 110. Also, the physiological signal measured by the physiological signal measuring apparatus is transmitted through the physiological signal transmission module 130 to an external device.

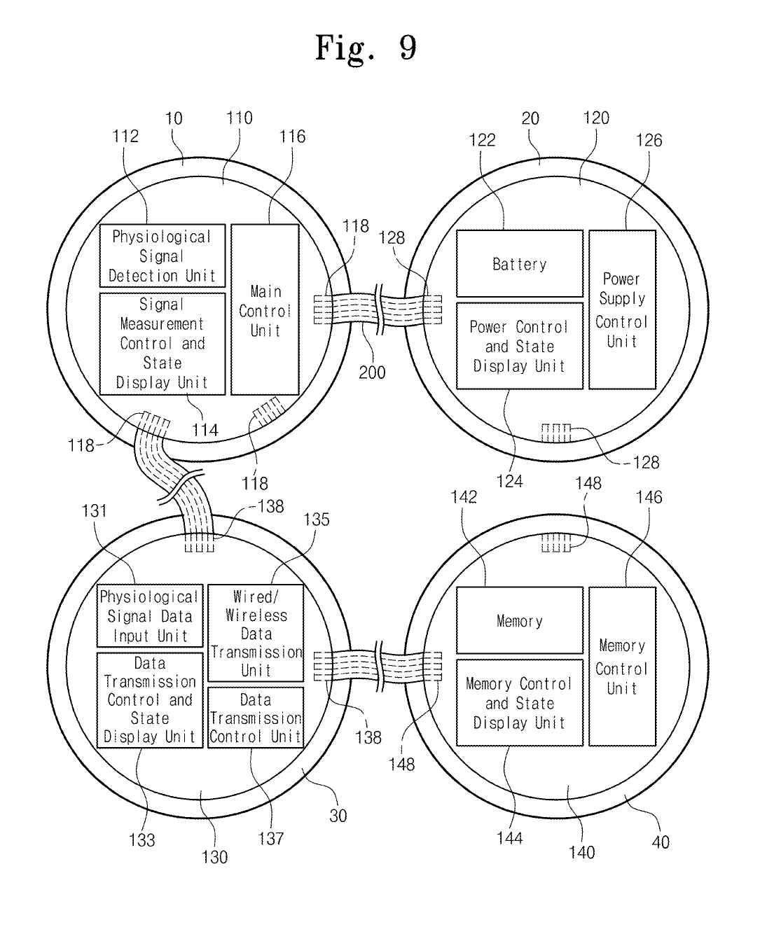

FIG. 8 illustrates an outline structure of a physiological signal measuring apparatus according to still another exemplary embodiment of the present invention. FIG. 9 illustrates a structure of each of the distributed modules of the physiological signal measuring apparatus according to still another exemplary embodiment of the present invention.

Referring to FIGS. 8 and 9, a physiological signal measuring apparatus according to still another exemplary embodiment of the present invention includes first to fourth detection electrodes 10, 20, 30 and 40, a physiological signal detection module 110, a power supply module 120, a physiological signal transmission module 130, and a physiological signal storage module 140.

The first to fourth detection electrodes 10, 20, 30 and 40, the physiological signal detection module 110, the power supply module 120, and the physiological signal transmission module 130 according to the exemplary embodiment of FIG. 8 are substantially identical to those according to the exemplary embodiment of FIGS. 1 and 5, and thus their description will be omitted for conciseness.

The physiological signal storage module 140 may be selectively connected through a connection line 200 to the physiological signal detection module 110, the power supply module 120, and the physiological signal transmission module 130. Also, the physiological signal storage module 140 may be connected to a plurality of other modules simultaneously.

The connection line 200, connecting the physiological signal storage module 140 and other module, includes a data line for communicating physiological signal data and a power line for supplying power. For example, the physiological signal storage module 140 receives physiological signal data and power from the physiological signal transmission module 130 through the connection line 200.

The physiological signal storage module 140 may store physiological signal data received from the physiological signal detection module 110. Specifically, the physiological signal storage module 140 includes a memory 142, a memory control and state display unit 144, a memory control unit 146, and one or more module connection ports 148. For example, the memory 142 may be a random access memory (RAM) and/or a flash memory, and may store physiological signal data.

The memory control and state display unit 144 may control data storage and stored data according to a user's command, and displays a residual memory capacity and an operation state of the physiological signal storage module 140.

The memory control unit 146 controls an operation of the physiological signal storage module 140. That is, the memory control unit 146 controls input/output of physiological signal data, the memory 142, and the memory control and state display unit 144.

A connection line 200 is connected to the module connection port 148 to provide electrical connection between the modules. The module connection port 148 includes a data input/output port (e.g., an RS 232 port or a USB port) for serial communication of physiological signal data and/or a power port. Power and physiological signal data may be inputted from the physiological signal transmission module 130 through the module connection port 148. Also, since the physiological signal storage module 140 has a plurality of module connection ports 148, other memory storage modules may be additionally connected through the module connection ports 148.

In another exemplary embodiment of the present invention, the physiological signal transmission module 130 may be omitted, and the physiological signal storage module 140 may be directly connected to the physiological signal detection module 110 through the connection line.

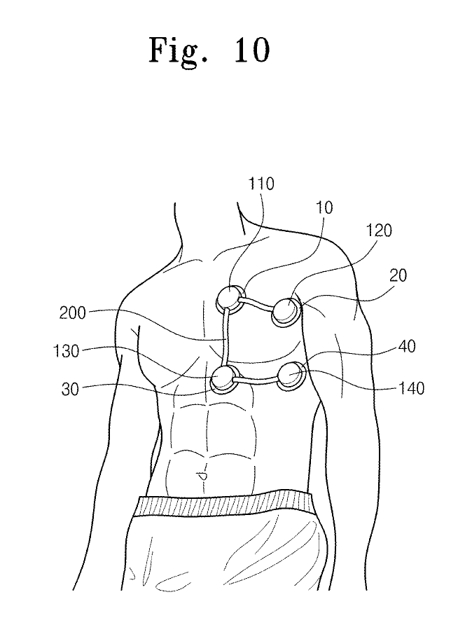

FIG. 10 illustrates an example of the use of the physiological signal measuring apparatus according to still another exemplary embodiment of the present invention.

Referring to FIG. 10, the physiological signal measuring apparatus can measure a 3-channel ECG when the first to fourth detection electrodes 10, 20, 30 and 40, at which the modules 110, 120, 130 and 140 are installed in a distributed manner, are attached to a heart portion of the human body. Herein, the first detection electrode 10, at which the physiological signal detection module 110 is installed, may be adhered around an SA node that serves as a source for generating a heart pulsation pulse.

When the physiological signal measuring apparatus is attached and the modules are operated, a physiological potential generated according to the shrinkage and expansion of heart muscles can be measured and a physiological signal can be displayed on the state display unit of the physiological signal detection module 110. Also, the physiological signal measured by the physiological signal measuring apparatus is transmitted through the physiological signal transmission module 130 to an external device. Also, in the long-time measurement of physiological signals, the physiological signal data generated by the physiological signal detection module 110 are stored in the physiological signal storage module 140.

Also, the connections between the modules 110, 120, 130 and 140 may vary in shape according to the bent or motion of the human body.

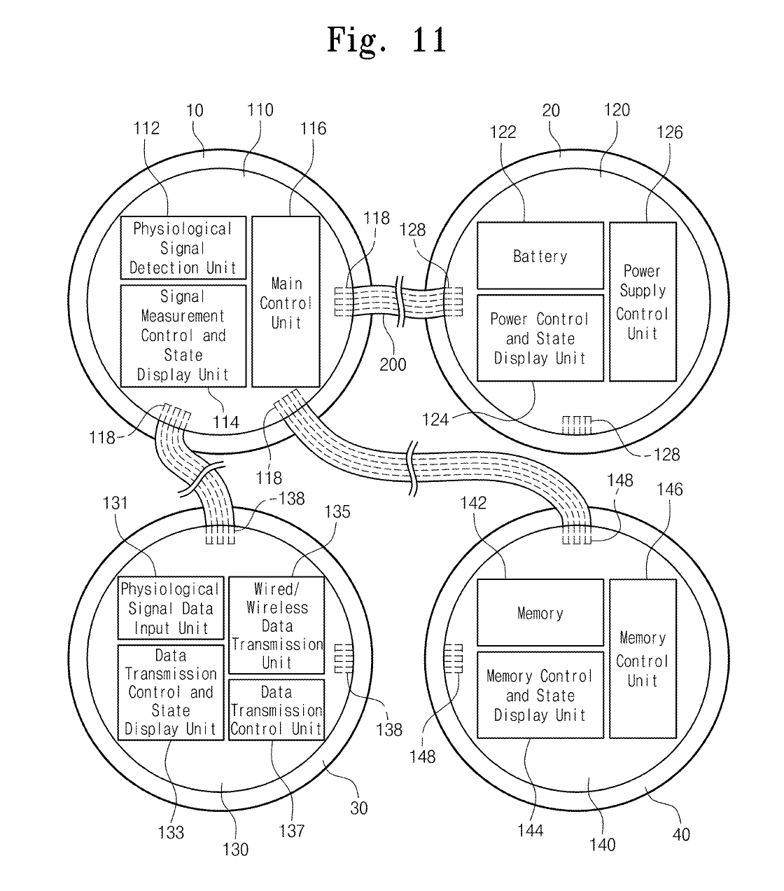

FIGS. 11 to 14 illustrate various connections between the modules in the physiological signal measuring apparatus according to exemplary embodiments of the present invention.

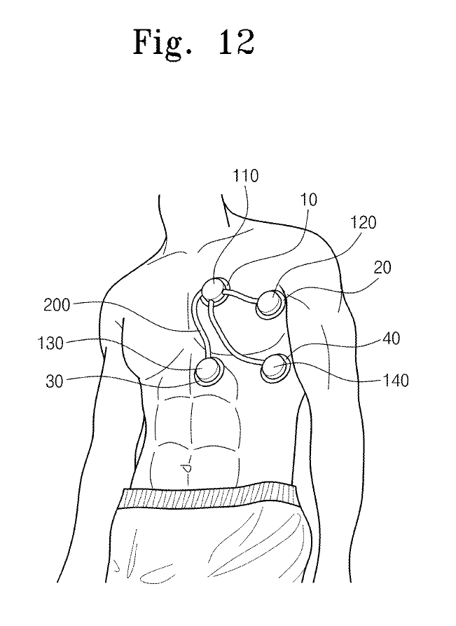

Referring to FIGS. 11 and 12, all of the power supply module 120, the physiological signal transmission module 130, and the physiological signal storage module 140 may be connected in common to the physiological signal detection module 110.

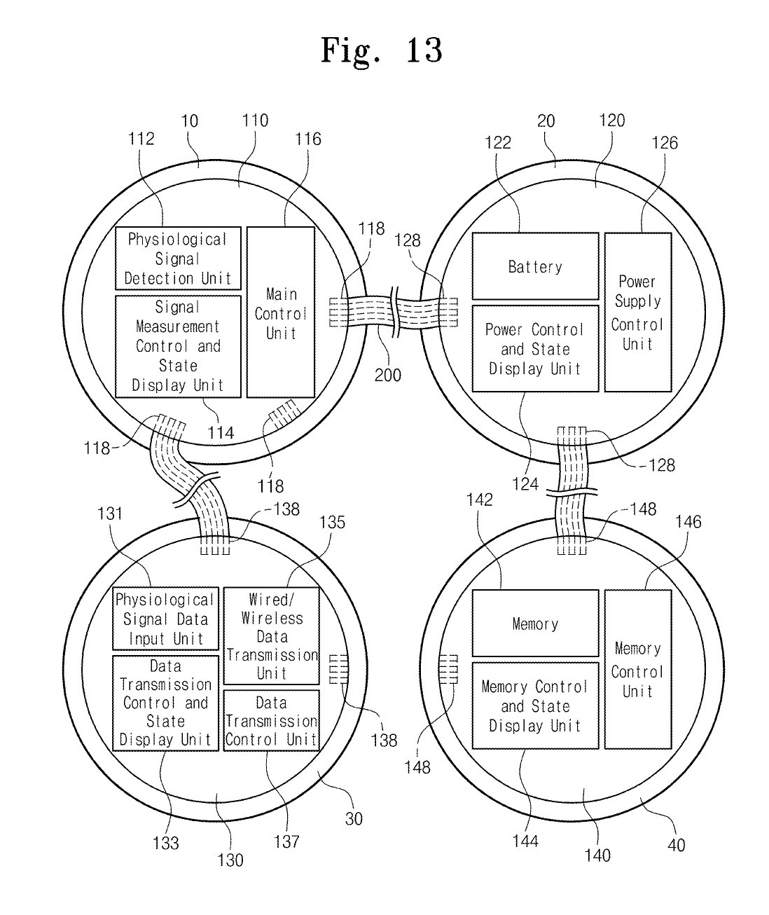

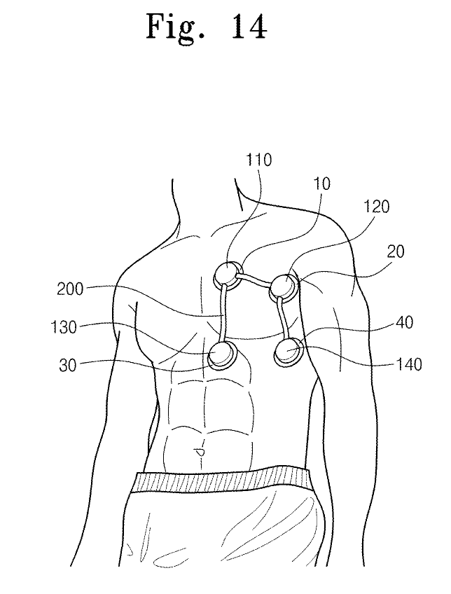

Also, referring to FIGS. 13 and 14, the physiological signal detection module 110 is connected through the connection line 120 to the power supply module 120 and the physiological signal transmission module 130. Also, the physiological signal detection module 110 may be directly connected through the connection line to the power supply module 120. In this case, power may be directly supplied through the power supply module 120 to the physiological signal storage module 140, and the physiological signal data generated by the physiological signal detection module 110 may be received through the power supply module 120.

As described above, according to the physiological signal measuring apparatuses of the present invention, modules for measuring physiological signals are distributed according to functions, thereby making it possible to reduce the concentration of a foreign-body sense on one point of the human body in the measurement of physiological signals. Therefore, the physiological signal measuring apparatus can be prevented from separating from the human body in the measurement of physiological signals, and the long-time measurement of physiological signals is easy to implement.

Also, since the modules are distributed according to functions, a memory for storing physiological signals in the measurement of physiological signals can be added or replaced and a battery can be replaced easily.

Also, since the modules of the physiological signal measuring apparatus are distributed, the sizes of the modules can be reduced.

The above-disclosed subject matter is to be considered illustrative and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An apparatus for measuring physiological signals, comprising:
first and second detection electrodes adhered to a target skin to detect physiological signals;
a physiological signal detection module installed at the first detection electrode to generate physiological signal data from the physiological signals detected by the first and second detection electrodes;
a power supply module installed at the second detection electrode to supply power to the physiological signal detection module; and
a connection line connecting the physiological signal detection module and the power supply module.

2. The apparatus of claim 1, wherein each of the physiological signal detection module and the power supply module comprises a display unit displaying an operation state of the corresponding module and a control unit allowing a user to control the corresponding module.

3. The apparatus of claim 1, wherein the physiological signal detection module comprises a plurality of module connection ports.

4. The apparatus of claim 3, wherein the module connection ports comprise a data input/output port and a power port.

5. The apparatus of claim 1, wherein the connection line has flexibility and elasticity.

6. The apparatus of claim 1, wherein the physiological signal detection module comprises:
an analog signal processing unit amplifying and filtering the physiological signal detected by the first detection electrode;
an analog-to-digital converting unit converting an analog signal of the physiological signal into a digital signal of the physiological signal data; and
a digital signal processing unit processing the physiological signal data through predetermined data operations.

7. The apparatus of claim 1, wherein the first and second detection electrodes have an adhesive material, which is adherable to a skin surface, on one surface and have a fixing member, at which the measurement module is installable, on the other surface.

8. The apparatus of claim 1, wherein the power supply module comprises a disposable battery or a rechargeable battery.

9. The apparatus of claim 1, wherein the physiological signals include electrocardiogram (ECG), electromyogram (EMG), electroencephalogram (EEG), and galvanic skin reflex (GSR).

10. The apparatus of claim 1, further comprising:
a third detection electrode adhered to a target skin to detect a physiological signal; and
a physiological signal transmission module installed at the third detection electrode to transmit the physiological signal data, which are detected by the physiological signal detection module, to an external device.

11. The apparatus of claim 10, wherein the physiological signal detection module generates the physiological signal data from the physiological signals detected by the first to third detection electrodes.

12. The apparatus of claim 10, wherein the physiological signal transmission module is connected to the physiological signal detection module and/or the power supply module.

13. The apparatus of claim 10, wherein the physiological signal transmission module comprises:
a display unit displaying an operation state of the module;
a control unit allowing the user to control the physiological signal transmission module;
a data input unit receiving the physiological signal data from the physiological signal detection module; and
a wired/wireless data transmission unit transmitting the physiological signal data to the external device through wired/wireless communication.

14. The apparatus of claim 10, wherein the physiological signal transmission module comprises a plurality of module connection ports.

15. The apparatus of claim 1, further comprising:
a fourth detection electrode adhered to a target skin to detect a physiological signal; and
a physiological signal storage module installed at the fourth detection electrode to store the physiological signal data detected by the physiological signal detection module.

16. The apparatus of claim 15, wherein the physiological signal detection module generates the physiological signal data from the physiological signals detected by the first to fourth detection electrodes.

17. The apparatus of claim 15, wherein the physiological signal storage module is connected to the physiological signal detection module and/or the power supply module.

18. The apparatus of claim 15, wherein the physiological signal storage module comprises:
a display unit displaying an operation state of the physiological signal storage module; and
a control unit allowing the user to control the physiological signal storage module.

19. The apparatus of claim 15, wherein the physiological signal storage module comprises a plurality of module connection ports.

20. The apparatus of claim 1, further comprising:
third and fourth detection electrodes adhered to a target skin to detect physiological signals;
a physiological signal transmission module installed at the third detection electrode to transmit the physiological signal data, which are detected by the physiological signal detection module, to an external device; and
a physiological signal storage module installed at the fourth detection electrode to store the physiological signal data detected by the physiological signal detection module,
wherein the physiological signal transmission module and the physiological signal storage module comprise a plurality of module connection ports.
